(19) United States
(12) Patent Application Publication
(10) Pub. No.: US 2026/0215915 A1
(43) Pub. Date: Jul. 30, 2026

(54) PROSTHETIC HEART VALVES INCLUDING FRAMES WITH INTEGRAL MARKERS

(71) Applicant: Edwards Lifesciences Corporation, Irvine, CA (US)

(72) Inventors: Rachel Christine Leacock, Torrance, CA (US); Ashley Akemi Ishigo, Long Beach, CA (US); Jennifer Marie Reitmajer, Costa Mesa, CA (US); Insiya Shakir Khambaty, Diamond Bar, CA (US); Anthony Michael Romero, Corona, CA (US); Venkateswaran Shanmugam, Lake Forest, CA (US)

(21) Appl. No.: 19/573,422

(22) Filed: Mar. 20, 2026

Related U.S. Application Data
(63) Continuation of application No. PCT/US2024/047386, filed on Sep. 19, 2024.
(60) Provisional application No. 63/584,160, filed on Sep. 20, 2023.

(51) Int. Cl. A61F 2/24; A61F 2/91

(52) U.S. Cl. CPC A61F 2/2418; A61F 2/91; A61F 2250/0098

(57) ABSTRACT

Prosthetic valves including a frame having one or more integral radiopaque markers and implantation and post-implantation methods. A prosthetic heart valve can comprise a frame including interconnected struts forming cells of the frame and a valve structure disposed in an interior of the frame. The valve structure includes a plurality of commissure where adjacent leaflets are joined at their outflow ends. Each of the commissures is attached to a commissure-supporting cell of the frame. Selected ones of the cells, such as commissure-supporting cells or cells adjacent to a commissure-supporting cells, can include an integral marker extending from an interior edge of the cell and into an open space of the cell. In some examples, the marker is a directional indicator. The marker can be configured to indicate a location of the selected cell during implantation procedures or post-implantation procedures while imaging the prosthetic valve via, for example, fluoroscopy.

### CROSS-REFERENCE TO RELATED APPLICATIONS

This application is a continuation of International Application No. PCT/US2024/047386, filed September 19, 2024, which claims the benefit of U.S. Patent Application No. 63/584,160, filed September 20, 2023, the entire disclosures all of which are incorporated by reference for all purposes.

### FIELD

The present disclosure relates to prosthetic heart valves including a frame having one or more integral markers for indicating a location on the frame, for example, for indicating a location and/or an orientation of a commissure-supporting cell of the frame.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient’s vasculature (for example, through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

In some examples, it may be desirable to visualize a location of at least one of the commissures of the prosthetic valve using fluoroscopy during the implantation procedure in order to implant the prosthetic heart valve in a desired rotational alignment relative to an anatomical landmark of the native valve. It some examples, it may be desirable to visualize a location of one or more of the commissures of the prosthetic valve during a post-implantation procedure, such as, when inserting a catheter into a coronary artery after implantation of the prosthetic valve.

### SUMMARY

Described herein are prosthetic heart valves, delivery apparatus, and methods for implanting prosthetic heart valves. The disclosed prosthetic heart valves, delivery apparatus, and methods can, for example, provide integral frame markers that can be utilized for identification of one or more cells of the frame of the prosthetic valve, such as one or more cells that each support a commissure of the prosthetic valve, and enable positioning of the prosthetic heart valve relative to a patient’s native anatomy and/or another implanted device. As such, the devices and methods disclosed herein can, among other things, overcome one or more of the deficiencies of typical prosthetic heart valves and their delivery apparatus.

A prosthetic heart valve can comprise a frame and a valve structure coupled to the frame. In addition to these components, a prosthetic heart valve can further comprise one or more of the components disclosed herein.

In some examples, the frame for the prosthetic heart valve can comprise a plurality of interconnected struts that form a plurality of cells including one or more commissure-supporting cells.

In some examples, the valve structure for the prosthetic heart valve can comprise a plurality of leaflets, each of the leaflets joined to an adjacent leaflet at the outflow end of the valve structure to form a commissure therebetween, the commissures attached to the frame at the commissure-supporting cells.

In some examples, a prosthetic heart valve can comprise a frame that includes an integral marker, the integral marker extending from an interior edge of one or more struts forming a cell of the frame and into an interior opening the cell.

In some examples, the cell is a commissure-supporting cell.

In some examples, the cell is adjacent to a commissure-supporting cell.

In some examples, the integral marker does not extend outside of the interior opening.

In some examples, the integral marker does not extend from an exterior edge of the cell.

In some examples, the integral marker can extend from the interior edge at an outflow apex portion of the cell.

In some examples, the integral marker can extend from the interior edge at a junction portion of two interconnected struts at an apex the cell.

In some examples, the integral marker can extend from the interior edge at a junction portion of two interconnected struts at one side of a cell.

In some examples, the integral marker can have an asymmetrical shape.

In some examples, the integral marker can have an asymmetrical orientation within a cell.

In some examples, the integral marker can include a head portion and a neck portion, the neck portion connected to a strut of the cell and the head portion extending into the open space of the cell.

In some examples, the integral marker can include a curved member, where first and second ends of the curved member are attached to one or more struts of the cell.

In some examples, the integral marker can be a directional indicator.

In some examples, the integral marker can be non-directional.

In some examples, the integral marker can be sized to enable axial elongation of a commissure-supporting cell when the frame is a radially compressed state.

In some examples, the integral marker can be shaped to enable axial elongation of a commissure-supporting cell when the frame is a radially compressed state.

In some examples, the integral marker can be disposed at an orientation to enable axial elongation of a commissure-supporting cell when the frame is a radially compressed state.

In some examples, the integral marker can be disposed at location within the cell to enable axial elongation of a commissure-supporting cell when the frame is a radially compressed state.

In some examples, the integral marker can be configured such that a width of the integral marker is less than or equal to a width of the commissure-supporting cell, when the cell is in the axially elongated state, at the location of the integral marker.

In some examples, a cell can include more than one integral marker.

In some examples, a cell can include a first integral marker and a second integral marker.

In some examples, the first integral marker is a symmetrical marker that bisects at least a portion of the cell.

In some examples, the second integral marker is a directional marker that has an asymmetrical shape and/or an asymmetrical orientation within the cell.

In some examples, the integral marker can be configured to enable identification of a commissure-supporting cell during one or more of an implantation procedure or a post-implantation procedure of a prosthetic heart valve.

In some examples, asymmetrically shaped integral markers can be configured to enable identification of one or more forward-most commissure-supporting cells relative to other commissure-supporting cells.

In some examples, asymmetrically oriented integral markers can be configured to enable identification of one or more forward-most commissure-supporting cells relative to other commissure-supporting cells.

In some representative example, a prosthetic heart valve can comprise: an annular frame comprising a plurality of interconnected struts that form a plurality of cells; and a valve structure disposed within an interior space of the frame and having an inflow end and an outflow end, the valve structure comprising a plurality of leaflets, each of the leaflets joined to an adjacent leaflet at the outflow end of the valve structure to form a commissure therebetween, the commissure attached to the frame at a commissure-supporting cell of the plurality of cells; wherein the commissure-supporting cell defines an interior opening; and wherein the frame comprises an integral marker, the integral marker extending from an interior edge of the commissure-supporting cell into the interior opening.

In another representative example, a prosthetic valve can comprise: an annular frame comprising a plurality of interconnected struts forming a plurality of cells, wherein each of the cells comprises an interior opening; and a valve structure disposed within an interior space of the frame and having an inflow end and an outflow end, the valve structure comprising a plurality of leaflets; wherein selected ones of the cells include an integral marker extending from an interior edge of the respective selected cell and into the interior opening thereof; wherein no portion of the integral marker extends outside of the interior opening; and wherein the integral marker is configured to enable identification of the respective selected cell during one or more of an implantation procedure or a post-implantation procedure.

In another representative example, a prosthetic valve can comprise: an annular frame comprising a plurality of interconnected struts forming a plurality of cells, wherein each of the cells comprises an interior opening; and a valve structure disposed within an interior space of the frame and having an inflow end and an outflow end, the valve structure comprising a plurality of leaflets; wherein selected ones of the cells include an integral marker extending from an interior edge of the respective selected cell and into the interior opening thereof; wherein no portion of the integral marker extends outside of the interior opening; and wherein the integral marker is configured to enable identification of the respective selected cell during one or more of an implantation procedure or a post-implantation procedure.

In another representative example, a prosthetic heart valve comprising: an annular frame comprising a plurality of interconnected struts forming a plurality of cells, wherein each of the cells comprises an interior opening; and a valve structure disposed within an interior space of the frame and having an inflow end and an outflow end, the valve structure comprising a plurality of leaflets; wherein selected ones of the cells include an integral marker extending from an interior edge of the respective selected cell and into the interior opening thereof; wherein no portion of the integral marker extends outside of the interior opening; and wherein the integral marker is configured to enable identification of the respective selected cell during one or more of an implantation procedure or a post-implantation procedure.

In another representative example, a prosthetic heart valve comprising: an annular frame comprising a plurality of interconnected struts forming a plurality of cells, wherein each of the cells comprises an interior opening; and a valve structure disposed within an interior space of the frame and having an inflow end and an outflow end, the valve structure comprising a plurality of leaflets; wherein selected ones of the cells each include one or more integral markers extending from an interior edge of the respective selected cell and into the interior opening thereof; wherein no portion of the integral marker extends outside of the interior opening; and wherein the one or more integral markers are configured to enable identification of the respective selected cells during one or more of an implantation procedure or a post-implantation procedure.

In some examples, a prosthetic heart valve comprises one or more of the components recited in Examples 1–32 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1A is a perspective view of a prosthetic heart valve, according to one example.

FIG. 1B is a perspective view of the prosthetic valve of FIG. 1A with the components on the outside of the frame shown in transparent lines for purpose of illustration.

FIG. 2 is a side view of an example of a delivery apparatus configured to deliver and implant a radially expandable prosthetic heart valve at an implantation site.

FIGS. 3A and 3B are enlarged side views of portions of frames including an exemplary integral marker extending from an interior edge of an outflow junction portion of a cell of the frame.

FIGS. 4A and 4B are enlarged side views of portions of frames including an exemplary integral marker extending from interior edges of two struts forming a cell of the frame.

FIGS. 5A and 5B are enlarged side views of portions of frames including an exemplary integral marker extending from an interior edge of a strut forming an outflow portion of a cell of the frame.

FIGS. 6A and 6B are enlarged side views of portions of frames including an exemplary integral marker extending from an interior edge of a side junction portion of a cell of the frame.

FIGS. 7A and 7B are enlarged side views of portions of frames including an exemplary integral marker extending from an interior edge of a strut forming an inflow portion of a cell of the frame.

FIGS. 8A-8C are side views of frames including exemplary integral markers, showing the frames in a radially compressed configuration.

FIGS. 9A and 9B are top perspective and front views of a frame including first and second exemplary integral markers.

FIGS. 10A and 10B are enlarged side views of portions of the frame of FIGS. 9A and 9B, respectively showing the frame in a radially expanded state and a radially compressed state, respectively.

FIG. 11 is a front view of a top portion of the frame of FIGS. 9A and 9B in a radially compressed state.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient’s body), while distal motion of the device is motion of the device away from the user and toward the implantation site (for example, into the patient’s body). The terms “longitudinal” and “axial” refer to an axis extending in the proximal and distal directions, unless otherwise expressly defined.

As used herein, “e.g.” means “for example,” and “i.e.” means “that is.”

### Overview of the Disclosed Technology

As introduced above, in some examples, it may be desirable to visualize a location of at least one of the commissures of the prosthetic valve using fluoroscopy during implantation procedures and/or post-implantation procedures. In some examples, a prosthetic valve can comprise a radiopaque marker secured to a portion of the prosthetic heart valve in order to allow visualization of the commissure under imaging (for example, fluoroscopy) during an implantation procedure or a post-implantation procedure.

For example, it may be desirable to visualize a location of one or more commissures of the prosthetic valve during an implantation procedure at a native valve in order to implant the prosthetic valve in a desired position relative to the native valve (such as, for example, a desired rotational alignment and/or axial position). In some examples, it may be desirable to implant the prosthetic valve such that commissures of the prosthetic valve align with commissures of the native valve or a previously implanted prosthetic valve. By including a radiopaque marker at one or more of the commissures of the prosthetic valve, a location of the commissures relative to the native valve (or other native anatomy or a previously implanted valve) can be visualized under medical imaging (for example, fluoroscopy) during the implantation procedure, thereby facilitating rotational positioning of the prosthetic valve relative to the native valve. As a result, a user can determine a location of the commissure relative to the native anatomy (native heart valve) and more easily implant the prosthetic valve in a desired rotational, axial, and/or lateral orientation relative to the native heart valve.

In another example, it may be desirable to visualize a location of one or more commissures of the prosthetic valve during a post-implantation procedure at a native valve having the prosthetic valve implanted therein in order to navigate a transcatheter instrument and/or an additional implantable device (such as, for example, a stent) through the implanted prosthetic valve. In some examples, it may be desirable to avoid commissures of the implanted prosthetic valve when navigating the transcatheter instrument and/or the additional implantable device to its target location (for example, to a coronary artery when the prosthetic valve is implanted in the native aortic valve). By including a radiopaque marker at one or more of the commissures of the prosthetic valve, a location of the commissures can be visualized under medical imaging (for example, fluoroscopy) during the post-implantation procedure, thereby facilitating navigation of the transcatheter instrument and/or the additional implantable device between the commissures of the implanted the prosthetic valve and into a coronary artery. As a result, a user can determine a location of the commissures of the implanted prosthetic valve and more easily perform the post-implantation procedure.

However, attachment of radiopaque markers to a frame or another portion of a prosthetic valve can require additional manufacturing steps, thereby increasing manufacturing time, complexity, and cost for the prosthetic valve. Further, the attachment of the radiopaque markers can be a source of error in manufacturing of the prosthetic valve (for example, where attachment fails or where attachment occurs at a location offset from a commissure or other target location).

The prosthetic valves and frames disclosed herein can address one or more of the foregoing issues. In some examples, a prosthetic valve includes a frame having one or more integral radiopaque markers formed on the frame. In some examples, integral markers are formed during the manufacturing of the frame (for example, cutting, molding, extruding, etc. of the frame). In some examples, the frame with one or more radiopaque markers is formed by laser cutting a metal tube. In some examples, an integral marker is formed within a cell of the frame. As used herein, a frame cell is an opening in the frame that is completely surrounded by one or more struts connected to each other to form a periphery or boundary surrounding the opening. In some examples, an integral marker extends from an interior edge of one or more struts forming a cell. In some examples, an integral marker is asymmetrical in order to function as a rotational directional indicator. In some examples, an integral marker is formed in a portion of the frame to indicate a location of a commissure-supporting cell. In some examples, an integral marker is formed within and extends into a commissure-supporting cell.

In some examples, the prosthetic valves disclosed herein can be radially compressible and expandable between a radially compressed state and a radially expanded state. In some examples, an integral radiopaque marker of the frame can be configured to enable radial compression of the frame when the prosthetic valve is in the radially compressed state. In other words, in such examples, the integral marker does not interfere with the radial compression of the frame when the prosthetic valve is in the radially compressed state.

Thus, the prosthetic valves can be crimped on or retained by an implant delivery apparatus in the radially compressed state while being advanced through a patient’s vasculature on the delivery apparatus. The prosthetic valve can be expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

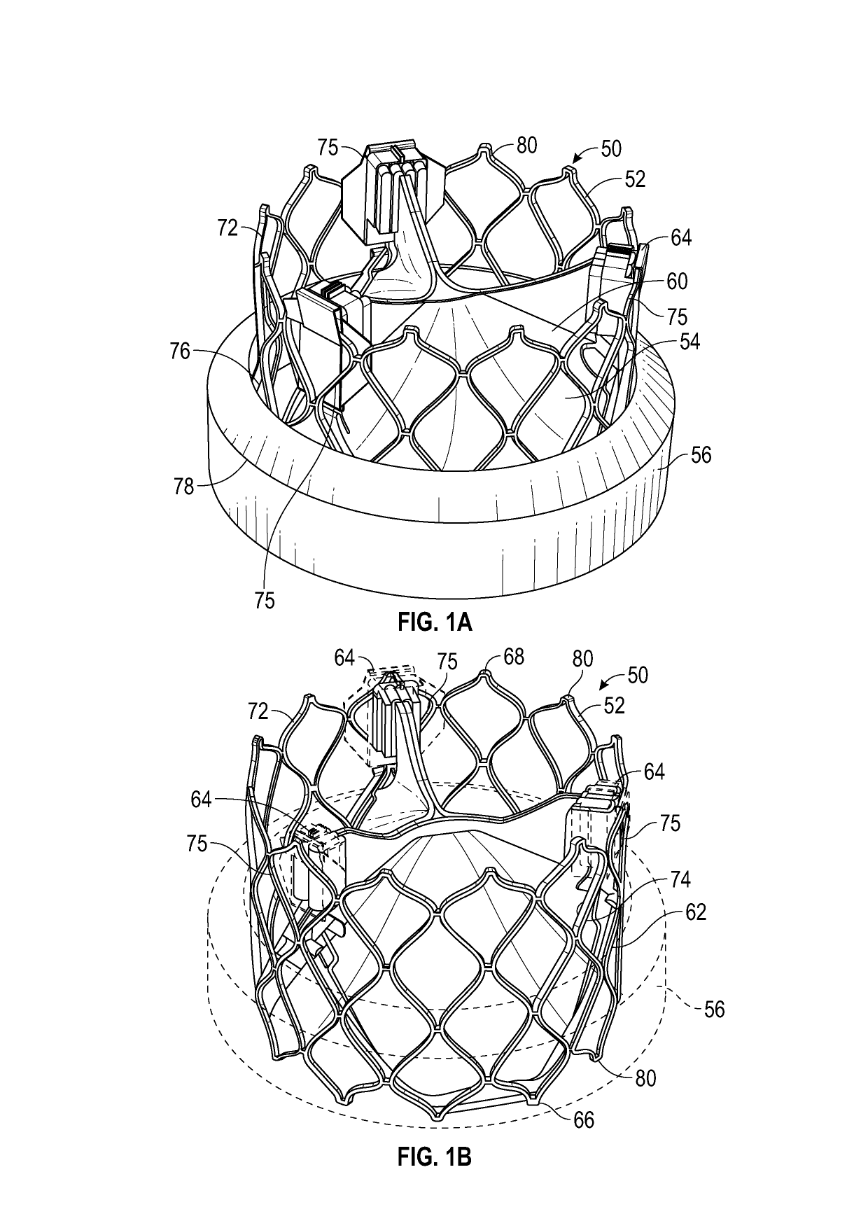

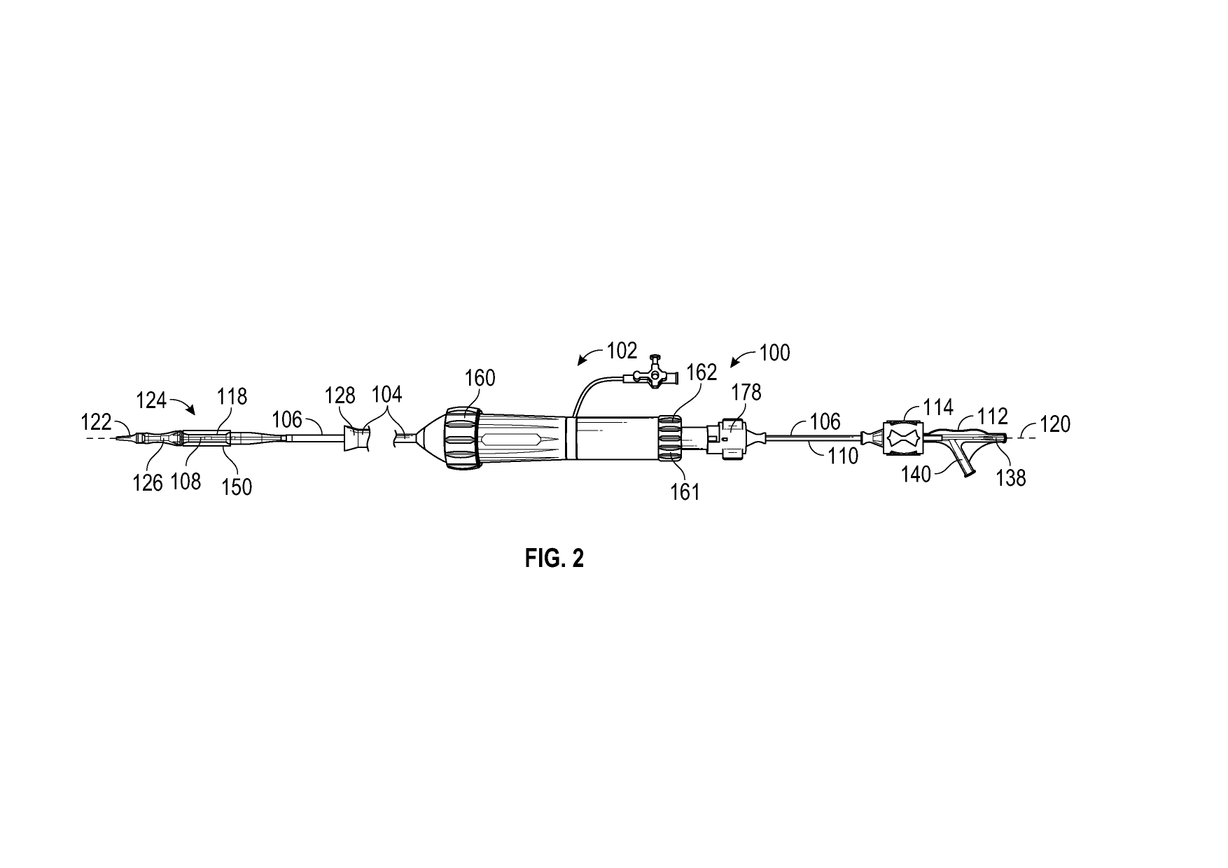

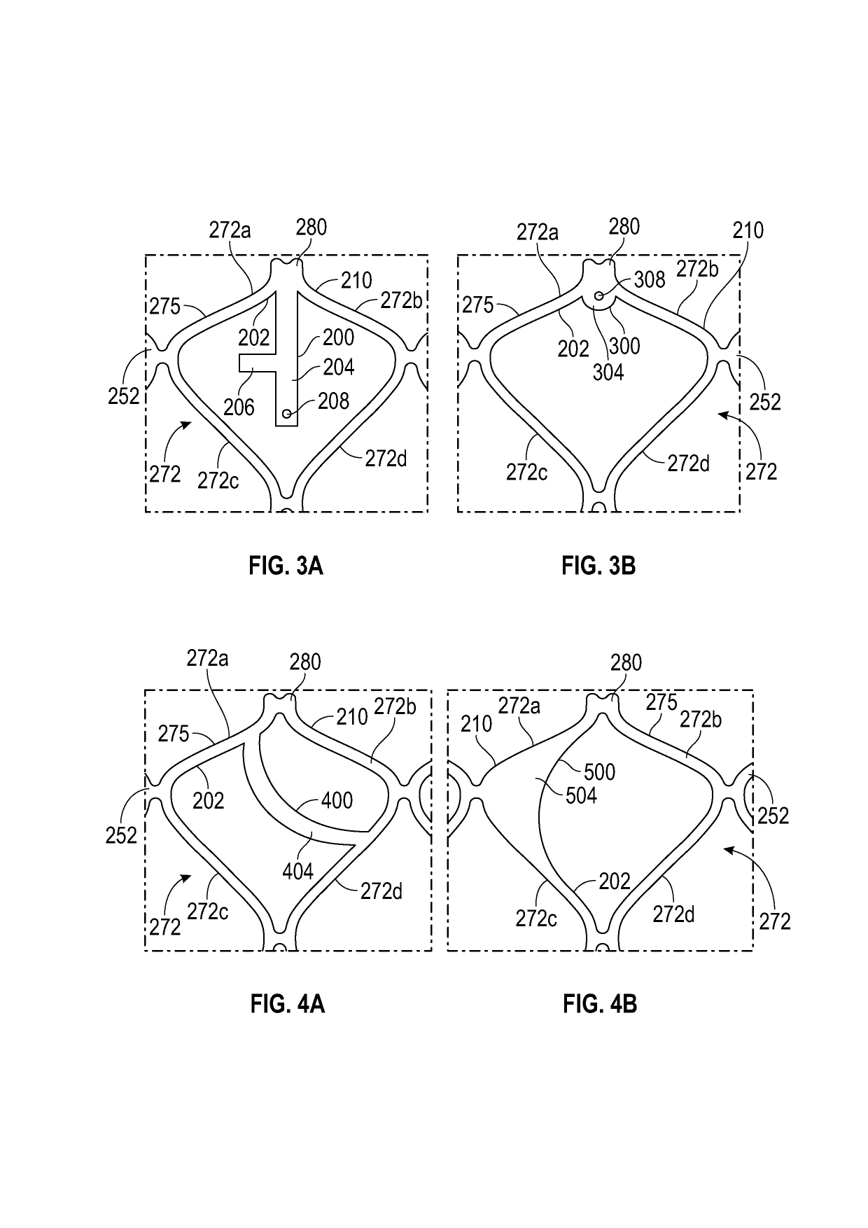

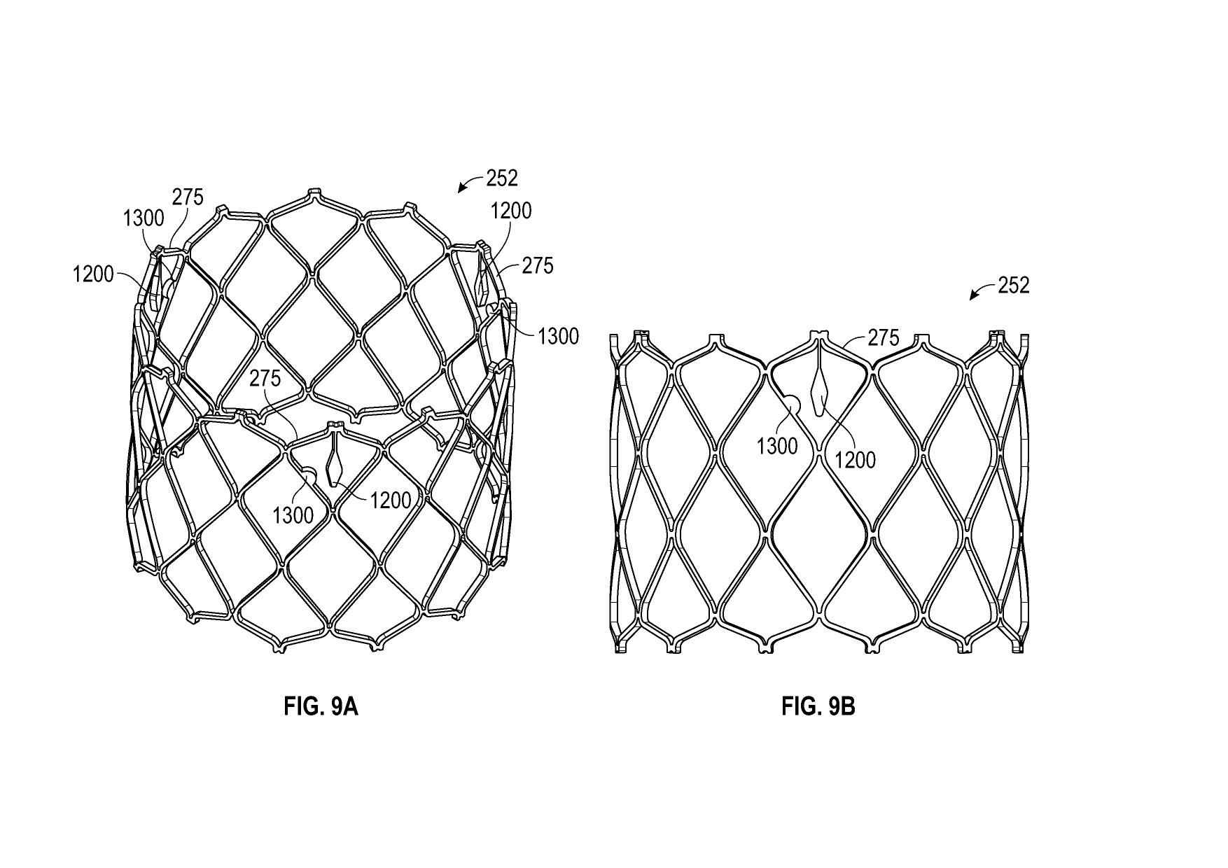

An exemplary prosthetic heart valve is shown in FIGS. 1A-1B. As introduced above, the prosthetic heart valve can be crimped on a delivery apparatus, such as the exemplary delivery apparatus shown in FIG. 2, in a radially compressed state while being advanced through a patient’s vasculature on the delivery apparatus. FIGS. 3A-7B and 9A-10A illustrate exemplary frames for a prosthetic heart valves including one or more integral markers, showing the frames in a radially expanded state. FIGS. 8A-8C and 10B-11 illustrate exemplary frames including one or more integral markers, showing the frames in a radially compressed state.

### Examples of the Disclosed Technology

FIGS. 1A and 1B show an exemplary prosthetic valve 50, according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

The prosthetic valve 50 can have three main components: a stent or frame 52, a valvular structure 54, and a sealing member 56 (FIG. 1A). FIG. 1B is a perspective view of the prosthetic valve 50 with the components on the outside of the frame 52 (including the sealing member 56) shown in transparent lines for purposes of illustration. The prosthetic valve 50 can have an inflow end 66 and an outflow end 68.

The frame 52 (and other frames disclosed here, such as the frame 252) can be made of any of various suitable plastically-expandable materials or self-expanding materials (for example, Nitinol). Suitable plastically-expandable materials that can be used to form the frame 52 include, metal alloys, polymers, or combinations thereof. Example metal alloys can comprise one or more of the following: nickel, cobalt, chromium, molybdenum, titanium, or other biocompatible metal. In some examples, the frame 52 can comprise stainless steel. In some examples, the frame 52 can comprise cobalt-chromium. In some examples, the frame 52 can comprise nickel-cobalt-chromium. In some examples, the frame 52 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

When constructed of a plastically-expandable material, the frame 52 (and thus the prosthetic valve 50) can be crimped to a radially collapsed configuration on a delivery apparatus (for example, a catheter) and then expanded inside a patient by an inflatable balloon or equivalent expansion mechanism. In some examples, the prosthetic valve 50 can be crimped directly onto the inflatable balloon of the delivery apparatus, such that the prosthetic valve 50 is axially aligned with and disposed radially outward of the balloon during advancing the prosthetic valve on the delivery apparatus to the implantation site, for example as described in PCT Application No. PCT/US2021/047056 (Published as WO2022/046585), which is incorporated herein by reference. In some examples, the prosthetic valve 50 can be crimped onto the delivery apparatus axially offset from the balloon, and then moved over the balloon at the implantation site, prior to inflation of the balloon and radial expansion of the prosthetic valve, such as described in U.S. Patent Application 9,339,384, which is incorporated herein by reference.

When constructed of a self-expandable material, the frame 52 (and thus the prosthetic valve 50) can be crimped to a radially collapsed configuration and restrained in the collapsed configuration by insertion into a sheath or equivalent mechanism of a delivery apparatus. Once inside the body, the prosthetic valve can be advanced from the delivery sheath, which allows the prosthetic valve to expand to its functional size.

Various crimping devices can be used to crimp the prosthetic valve 50 and the other prosthetic valves described herein around the delivery apparatus, such as the crimping devices described in U.S. Patent No. 7,530,253, which is incorporated herein by reference.

Returning to FIGS. 1A and 1B, the frame 52 in the illustrated example comprises a plurality of circumferentially extending rows of angled interconnected struts 72 defining rows of cells 74 defining openings of the frame. As shown in the illustrated example, each cell 74 is formed by interconnected struts 72, which form a continuous, closed boundary completely surrounding the opening. In some examples, the frame 52 can have a cylindrical or substantially cylindrical shape having a constant diameter from the inflow end 66 to the outflow end 68 of the frame 52 as shown. In some examples, the frame 52 can vary in diameter along the height of the frame, as disclosed in U.S. Patent Publication No. 2012/0239142, which is incorporated herein by reference.

The frame 52, at each of the inflow end 66 and the outflow end 68, may comprise a plurality of apices 80 spaced apart from one another around a circumference of the frame 52.

The valvular structure 54 can comprise three leaflets 60, collectively forming a leaflet structure, which can be arranged to collapse in a tricuspid arrangement, although in other examples there can be greater or fewer number of leaflets (for example, one or more leaflets 60). In some examples, the leaflets 60 can be formed of pericardial tissue (for example, bovine pericardial tissue), biocompatible synthetic materials, or various other suitable natural or synthetic materials as known in the art and described in U.S. Patent No. 6,730,118, which is incorporated by reference herein.

Each leaflet 60 can be coupled to the frame 52 along its inflow edge 62 (the lower edge in the figures; also referred to as “cusp edges”) and at commissures 64 of the valvular structure 54 where adjacent portions (for example, commissure tabs) of two leaflets are connected to each other. In some examples, the commissures 64 can comprise an attachment member (for example, comprising fabric, flexible polymer, or the like) arranged across a cell to form a commissure-supporting cell 75 of the frame 52. The attachment member can be secured to the interconnected struts 72 of the commissure-supporting cell 75 and the adjacent portions of the two leaflets can be connected to the attachment member to form the commissure 64. The commissure tabs and/or the attachment member can be secured to the struts forming the commissure-supporting cell 75, such as with sutures forming whip stitches extending through the commissure tabs (and/or the attachment member) and around the struts 72.

In some examples, a reinforcing element or connecting skirt, such as a fabric strip, can be connected directly to the cusp edges of the leaflets and to the struts of the frame to couple the cusp edges of the leaflets to the frame.

The sealing member 56 in the illustrated example is mounted on the outside of the frame 52 and functions to create a seal against the surrounding tissue (for example, the native leaflets and/or native annulus) to prevent or at least minimize paravalvular leakage. The sealing member 56 can comprise an inner layer 76 (which can be in contact with the outer surface of the frame 52) and an outer layer 78. The sealing member 56 can be connected to the frame 52 using suitable techniques or mechanisms. For example, the sealing member 56 can be sutured to the frame 52 via sutures that can extend around the struts 72 and through the inner layer 76. In alternative examples, the inner layer 76 can be mounted on the inner surface of the frame 52, while the outer layer 78 is on the outside of the frame 52.

The outer layer 78 can be configured or shaped to extend radially outward from the inner layer 76 and the frame 52 when the prosthetic valve 50 is deployed. When the prosthetic valve is fully expanded outside of a patient’s body, the outer layer 78 can expand away from the inner layer 76 to create a space between the two layers. Thus, when implanted inside the body, this allows the outer layer 78 to expand into contact with the surrounding tissue.

The sealing member 56 can be wholly or partly formed of any suitable biological material, synthetic material (for example, any of various polymers), or combinations thereof. In some examples, the skirt can comprise a fabric having interlaced yarns or fibers, such as in the form of a woven, braided, or knitted fabric. In some examples, the fabric can have a plush nap or pile. Exemplary fabrics having a plush nap or pile include velour, velvet, velveteen, corduroy, terrycloth, fleece, etc. In some examples, the sealing member can comprise a fabric without interlaced yarns or fibers or randomly interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide etc. In some examples, the sealing member can comprise a non-textile or non-fabric material, such as a film made from any of a variety of polymeric materials, such as PTFE, PET, polypropylene, polyamide, polyetheretherketone (PEEK), polyurethane (such as thermoplastic polyurethane (TPU)), etc. In some examples, the sealing member can comprise a sponge material or foam, such as polyurethane foam. In some examples, the sealing member can comprise natural tissue, such as pericardium (for example, bovine pericardium, porcine pericardium, equine pericardium, or pericardium from other sources).

Additional details regarding the prosthetic valve 50 and its various components are described in PCT Applicant No. PCT/US2021/047056 and U.S. Patent Publication No. 2018/0028310, which is incorporated herein by reference.

FIG. 2 shows a delivery apparatus 100, according to an example, that can be used to implant an expandable prosthetic heart valve 150 (for example, the prosthetic valve 50 of FIGS. 1A and 1B and/or any of the other prosthetic heart valves described herein). In some examples, the delivery apparatus 100 is specifically adapted for use in introducing a prosthetic valve into a heart.

The delivery apparatus 100 in the illustrated example of FIG. 2 is a balloon catheter comprising a handle 102 and a steerable, outer shaft 104 extending distally from the handle 102. The delivery apparatus 100 can further comprise an intermediate shaft 106 (which also may be referred to as a balloon shaft) that extends proximally from the handle 102 and distally from the handle 102, the portion extending distally from the handle 102 also extending coaxially through the outer shaft 104. Additionally, the delivery apparatus 100 can further comprise an inner shaft 108 extending distally from the handle 102 coaxially through the intermediate shaft 106 and the outer shaft 104 and proximally from the handle 102 coaxially through the intermediate shaft 106.

The outer shaft 104 and the intermediate shaft 106 can be configured to translate (for example, move) longitudinally, along a central longitudinal axis 120 of the delivery apparatus 100, relative to one another to facilitate delivery and positioning of a prosthetic valve at an implantation site in a patient’s body.

The intermediate shaft 106 can include a proximal end portion 110 that extends proximally from a proximal end of the handle 102, to an adaptor 112. A rotatable knob 114 can be mounted on the proximal end portion 110 and can be configured to rotate the intermediate shaft 106 around the central longitudinal axis 120 and relative to the outer shaft 104.

The adaptor 112 can include a first port 138 configured to receive a guidewire therethrough and a second port 140 configured to receive fluid (for example, inflation fluid) from a fluid source. The second port 140 can be fluidly coupled to an inner lumen of the intermediate shaft 106.

The intermediate shaft 106 can further include a distal end portion that extends distally beyond a distal end of the outer shaft 104 when a distal end of the outer shaft 104 is positioned away from an inflatable balloon 118 of the delivery apparatus 100. A distal end portion of the inner shaft 108 can extend distally beyond the distal end portion of the intermediate shaft 106.

The balloon 118 can be coupled to the distal end portion of the intermediate shaft 106.

In some examples, a distal end of the balloon 118 can be coupled to a distal end of the delivery apparatus 100, such as to a nose cone 122 (as shown in FIG. 2), or to an alternate component at the distal end of the delivery apparatus 100 (for example, a distal shoulder). An intermediate portion of the balloon 118 can overlay a valve mounting portion 124 of a distal end portion of the delivery apparatus 100 and a distal end portion of the balloon 118 can overly a distal shoulder 126 of the delivery apparatus 100. The valve mounting portion 124 and the intermediate portion of the balloon 118 can be configured to receive a prosthetic heart valve in a radially compressed state. For example, as shown schematically in FIG. 2, a prosthetic heart valve 150 (which can be one of the prosthetic valves described herein) can be mounted around the balloon 118, at the valve mounting portion 124 of the delivery apparatus 100.

The balloon shoulder assembly, including the distal shoulder 126, is configured to maintain the prosthetic heart valve 150 (or other medical device) at a fixed position on the balloon 118 during delivery through the patient’s vasculature.

The outer shaft 104 can include a distal tip portion 128 mounted on its distal end. The outer shaft 104 and the intermediate shaft 106 can be translated axially relative to one another to position the distal tip portion 128 adjacent to a proximal end of the valve mounting portion 124, when the prosthetic valve 150 is mounted in the radially compressed state on the valve mounting portion 124 (as shown in FIG. 2) and during delivery of the prosthetic valve to the target implantation site. As such, the distal tip portion 128 can be configured to resist movement of the prosthetic valve 150 relative to the balloon 118 proximally, in the axial direction, relative to the balloon 118, when the distal tip portion 128 is arranged adjacent to a proximal side of the valve mounting portion 124.

An annular space can be defined between an outer surface of the inner shaft 108 and an inner surface of the intermediate shaft 106 and can be configured to receive fluid from a fluid source via the second port 140 of the adaptor 112. The annular space can be fluidly coupled to a fluid passageway formed between the outer surface of the distal end portion of the inner shaft 108 and an inner surface of the balloon 118. As such, fluid from the fluid source can flow to the fluid passageway from the annular space to inflate the balloon 118 and radially expand and deploy the prosthetic valve 150.

An inner lumen of the inner shaft can be configured to receive a guidewire therethrough, for navigating the distal end portion of the delivery apparatus 100 to the target implantation site.

The handle 102 can include a steering mechanism configured to adjust the curvature of the distal end portion of the delivery apparatus 100. In the illustrated example, for example, the handle 102 includes an adjustment member, such as the illustrated rotatable knob 160, which in turn is operatively coupled to the proximal end portion of a pull wire. The pull wire can extend distally from the handle 102 through the outer shaft 104 and has a distal end portion affixed to the outer shaft 104 at or near the distal end of the outer shaft 104. Rotating the knob 160 can increase or decrease the tension in the pull wire, thereby adjusting the curvature of the distal end portion of the delivery apparatus 100. Further details on steering or flex mechanisms for the delivery apparatus can be found in U.S. Patent No. 9,339,384, which is incorporated by reference herein.

The handle 102 can further include an adjustment mechanism 161 including an adjustment member, such as the illustrated rotatable knob 162, and an associated locking mechanism including another adjustment member, configured as a rotatable knob 178. The adjustment mechanism 161 is configured to adjust the axial position of the intermediate shaft 106 relative to the outer shaft 104 (for example, for fine positioning at the implantation site). Further details on the delivery apparatus 100 can be found in PCT Application No. PCT/US2021/047056, which is incorporated by reference herein.

As discussed above, prosthetic heart valves disclosed herein can include a frame having one or more integral markers. In some examples, integral markers are formed during the manufacturing of the frame (for example, cutting, molding, extruding, etc. of the frame). In some examples, an integral marker is formed within a cell of the frame (that is, the integral marker extends into the opening bounded by interconnected struts that make up the cell) and can extend from an interior edge of one or more of the interconnected struts forming the cell. For example, an integral marker can be formed within and extend into a commissure-supporting cell so that the commissure-supporting cell can be identified (for example, visualized via fluoroscopy) during an implantation or a post-implantation procedure.

FIGS. 3A-11 illustrate exemplary integral markers for a frame of a prosthetic valve. Specifically, FIGS. 3A-11 show a cell 275 formed by interconnected struts 272 in an outflow row of cells of a frame 252, which can be a selected cell in the outflow row, such as a commissure-supporting cell of the frame or a cell adjacent to a commissure-supporting cell. In some examples, the frame 252 in FIGS. 3A-11 can be the same as the frame 52 and the cell 275 can be the same as one of the cells 75 of the frame 52, except for the addition of one or more integral markers (for example, integral markers 200, 300, 400, 500, 600, 700, 800, 900, 1000, 1100, 1200, 1300). Thus, in some examples, a prosthetic valve 50 can comprise the frame 252 (with any of the disclosed marker configurations) and any or all the remaining components shown or described in connection with FIGS. 1A-1B.

As shown in FIGS. 3A-11, the cell 275 can include one or more integral radiopaque marker (one or more of the integral radiopaque markers 200, 300, 400, 500, 600, 700, 800, 900, 1000, 1100, 1200, 1300) extending from an interior edge 202 of one or more of the struts 272 forming the cell and into the interior space or opening of the cell. In some examples, no portion or segment of the marker(s) extends outside of the cell. That is, in some examples, the marker(s) can extend only from the interior edge(s) of a cell (that is, from one or more interior edges of struts forming the cell). In some examples, the marker(s) can additionally include a portion that extends from an exterior edge of the cell. In some examples, a marker or combination of markers can include an asymmetrical component or configuration that is indicative of directionality. For example, a marker can be oriented within the cell to give the cell an overall asymmetrical shape (that is, the marker can have an asymmetrical orientation) and/or the marker itself can have an asymmetrical shape. In some examples, a marker can be symmetrical and/or can have a symmetrical orientation within the cell. In some examples, a marker can be configured to indicate (that is, enable identification of) a location of the selected cell during one or more of an implantation procedure or a post-implantation procedure.

In some examples, multiple cells in an outflow row of the frame 252 can include one or more radiopaque markers. For example, an outflow row can have three cells including each one or more integral markers, where the cells are circumferentially spaced relative to each other, such as being 120 degrees relative to each other in the outflow row. In some examples, the outflow row can include additional or fewer cells including one or more integral markers. In some examples, other cells of the frame, for example, one or more cells in an outflow row, can include one or more integral markers.

In some examples, an integral marker can extend from an interior edge at an apex portion of a cell in a frame. For example, FIG. 3A shows the cell 275 including a marker 200. The marker 200 can extend from the interior edge 202 at a junction between struts 272a and 272b, and can be disconnected from other struts of the cell (272c, 272d). In some examples, the junction between struts 272a and 272b can be an apex 280 of the frame 252. As illustrated in FIG. 3A, in the present example, no portion of the marker 200 extends from an exterior edge 210 of the cell 275. The marker 200 can include a main body comprising a linear member 204, which can extend parallel to a central axis of the frame and/or can bisect at least a portion of the cell 275. The marker 200 can additionally include a projection (for example, a tab portion) 206 that and projects outwardly from member 204 toward one side of the cell 275 (for example, at an orientation that is perpendicular to the member 204. The tab portion 206 can give the marker 200 an overall asymmetrical shape. Thus, the marker 200 can be a directional indicator (indicating a left or a right-handed direction of the frame 252). In some examples, the marker 200 can exclude the tab portion and can include only the linear member 204, thereby making the marker symmetrical (or non-directional). In some examples, the marker 200 can additionally include an aperture 208 at a free end of the linear member 204 (that is, at an opposing end of the member 204 relative to where it extends from the interior edge 202).

In another example, FIG. 3B shows the cell 275 including an integral marker 300. The marker 300 can extend from the interior edge 202 at a junction between struts 272a and 272b, and can be disconnected from other struts of the cell (272c, 272d). In some examples, the junction between struts 272a and 272b can be the apex 280 of the frame 252. As illustrated in FIG. 3B, no portion of the marker extends from the exterior edge 210 of the cell 275. The marker 300 can include a main body comprising a partial or semi-circular member 304 (for example, a 2/3 portion of a circle) having a continuously curved outer edge extending from the strut 272a to the strut 272b. In some examples, the marker 300 can additionally include an aperture 308 at a center portion of the circular member 304. In the present example, the marker 300 has an overall symmetrical shape and is thereby non-directional relative to a left or a right-handed direction of the frame 252. In other examples, the marker 300 can have a directional component or configuration. For example, the marker 300 can additionally include a projection (for example, a tab or other projection), or can have a bulging shape directed toward one side of the cell 275, giving the marker 300 an overall asymmetrical shape.

In some examples, an integral marker can extend from an interior edge of two or more struts of a cell of a frame, outside of an apex portion of the cell. For example, FIG. 4A shows the cell 275 including an integral marker 400. The marker 400 can extend from the interior edges 202 of each of the struts 272a and 272d, and can be disconnected from other struts of the cell (272b, 272c). As illustrated in FIG. 4A, in the present example, no portion of the marker extends from the exterior edge 210 of the cell 275. The marker 400 can include a main body comprising a curved member 404, which can extend across the open space of the cell 275 from the strut 272a (in an outflow portion of the cell) to the strut 272b (in an inflow portion of the cell). The curved member 404 can curve away from the strut 272b and toward the strut 272c, thereby giving the marker 400 an asymmetrical orientation within the cell 275. Thus, the marker 400 can be a directional indicator (indicating a left or a right-handed direction of the frame 252).

In another example, FIG. 4B shows the cell 275 including an integral marker 500. The marker 500 can extend from the interior edge 202 of the struts 272a and 272c, and can be disconnected from other struts of the cell (272b, 272d). As illustrated in FIG. 4B, in the present example, no portion of the marker extends from an exterior edge 210 of the cell 275. The marker 500 can include a main body comprising a crescent-shaped member 504 that is continuous with a junction portion of the struts 272a, 272c. In other words, the marker 500 can be formed by the struts 272a, 272c having a greater width at a junction portion of the struts 272a, 272c, which creates an asymmetrical orientation of the marker 500 within the cell 275. Thus, the marker 400 can be a directional indicator (indicating a left or a right-handed direction of the frame 252).

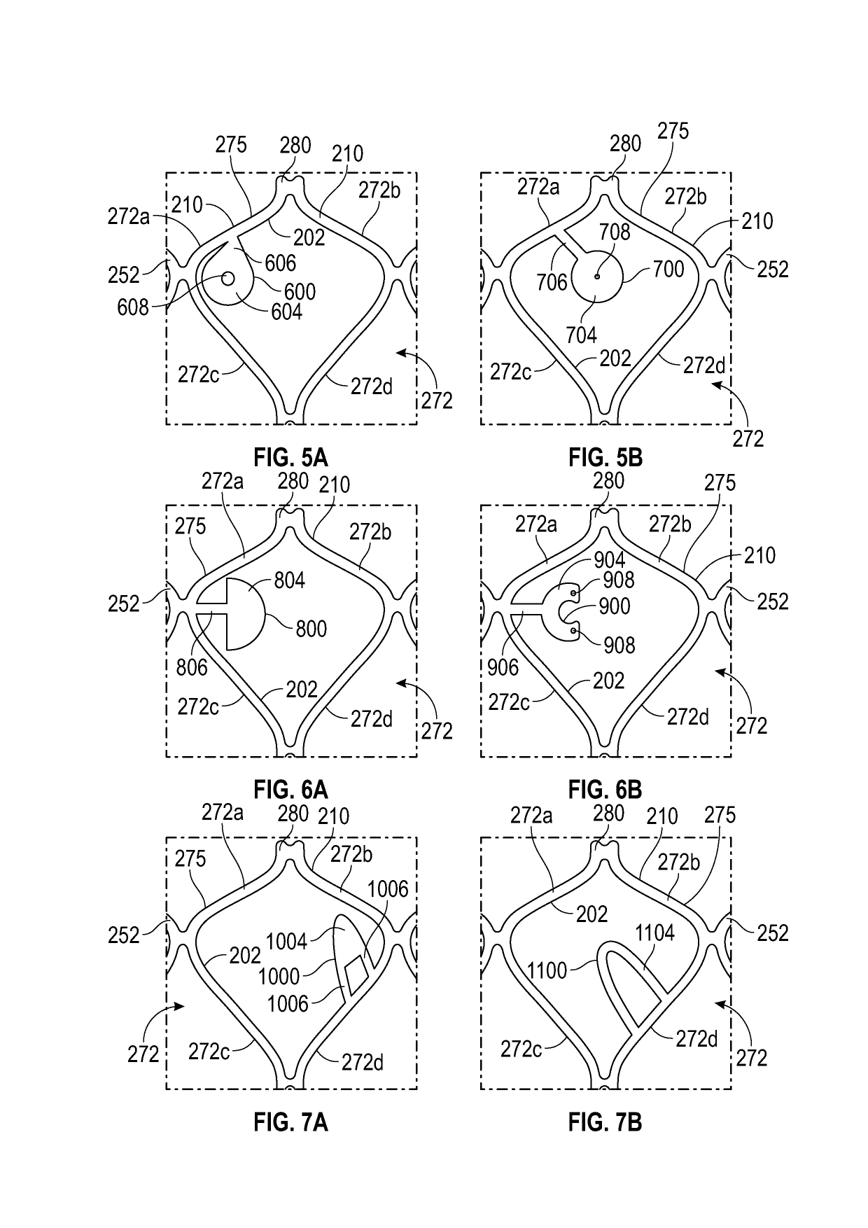

In some examples, an integral marker can extend from an interior edge of a strut in an outflow portion of a cell (that is, a portion of the cell oriented toward the outflow end of the prosthetic valve). For example, FIG. 5A shows the cell 275 including an integral marker 600. The marker 600 can extend from the interior edge 202 of the strut 272a, which is in an outflow portion of the cell 275, and can be disconnected from other struts of the cell (272b, 272c, 272d). As illustrated in FIG. 5A, in the present example, no portion of the marker extends from an exterior edge 210 of the cell 275. The marker 600 can include a main body comprising a circular member 604. The marker 600 can further include a V-shaped neck portion 606, wherein the V-shaped neck portion 606 extends between the interior edge 202 and the circular member 604, the narrowest portion thereof oriented towards the interior edge 202. The marker 600 can additionally include an aperture 608 in a center of circular member 604. The circular member 604 can extend into one side of the open area the cell 275 near a junction portion between the struts 272a, 272c, thereby giving the marker 600 an asymmetrical orientation within the cell 275. Thus, the marker 600 can be a directional indicator (indicating a left or a right-handed direction of the frame 252).

In another example, FIG. 5B shows the cell 275 including an integral marker 700. The marker 700 can extend from the interior edge 202 of the strut 272a, which is in the outflow end or portion of the cell 275, and can be disconnected from other struts of the cell (272b, 272c, 272d). As illustrated in FIG. 5B, in the present example, no portion of the marker 700 extends from the exterior edge 210 of the cell 275. The marker 700 can include a main body comprising a circular member 704. The marker 700 can further include a linear neck portion 706 that is narrower than the main body, wherein the linear neck portion 706 extends between the interior edge 202 and the circular member 704. The marker 700 can additionally include an aperture 708 in a center of circular member 704. The circular member 704 can extend into the open area the cell 275 at an angle toward a center of the cell, thereby giving the marker 700 an asymmetrical orientation within the cell 275. Thus, the marker 700 can be a directional indicator (indicating a left or a right-handed direction of the frame 252).

In some examples, an integral marker can extend from an interior edge of a side portion of a cell (that is, an interior edge at a junction between struts at one side of the cell). For example, FIG. 6A shows the cell 275 including an integral marker 800. The marker 800 can extend from the interior edge 202 at a junction portion of the struts 272a, 272b located at a side of the cell 275. As illustrated in FIG. 6A, in the present example, no portion of the marker 800 extends from an exterior edge 210 of the cell 275. The marker 800 can include a main body comprising a partial circular member 804 (for example, a half circle member). The marker 800 can further include a linear neck portion 806 that is narrower than the main body, wherein the neck portion 806 extends from the interior edge 202 to a flat region of the partial circular member 804. The partial circular member 804 can extend into one side of the open area the cell 275 near the junction portion between the struts 272a, 272c, thereby giving the marker 800 an asymmetrical orientation within the cell 275. Thus, the marker 800 can be a directional indicator (indicating a left or a right-handed direction of the frame 252).

In another example, FIG. 6B shows the cell 275 including an integral marker 900. The marker 900 can extend from the interior edge 202 at a junction portion of the struts 272a, 272b located at a side of the cell 275. As illustrated in FIG. 5B, in the present example, no portion of the marker extends from an exterior edge 210 of the cell 275. The marker 900 can include a main body comprising a C-shaped member 904. The marker 900 can further include a linear neck portion 806 that is narrower than the main body, wherein the neck portion 906 extends from the interior edge 202 to a center of an outer curve of the C-shaped member 904. The marker 900 can additionally include apertures 908 in opposing ends of the C-shaped member 904. The C-shaped member 904 can extend into one side of the open area the cell 275 near the junction portion between the struts 272a, 272c, thereby giving the marker 900 an asymmetrical orientation within the cell 275. Thus, the marker 900 can be a directional indicator (indicating a left or a right-handed direction of the frame 252).

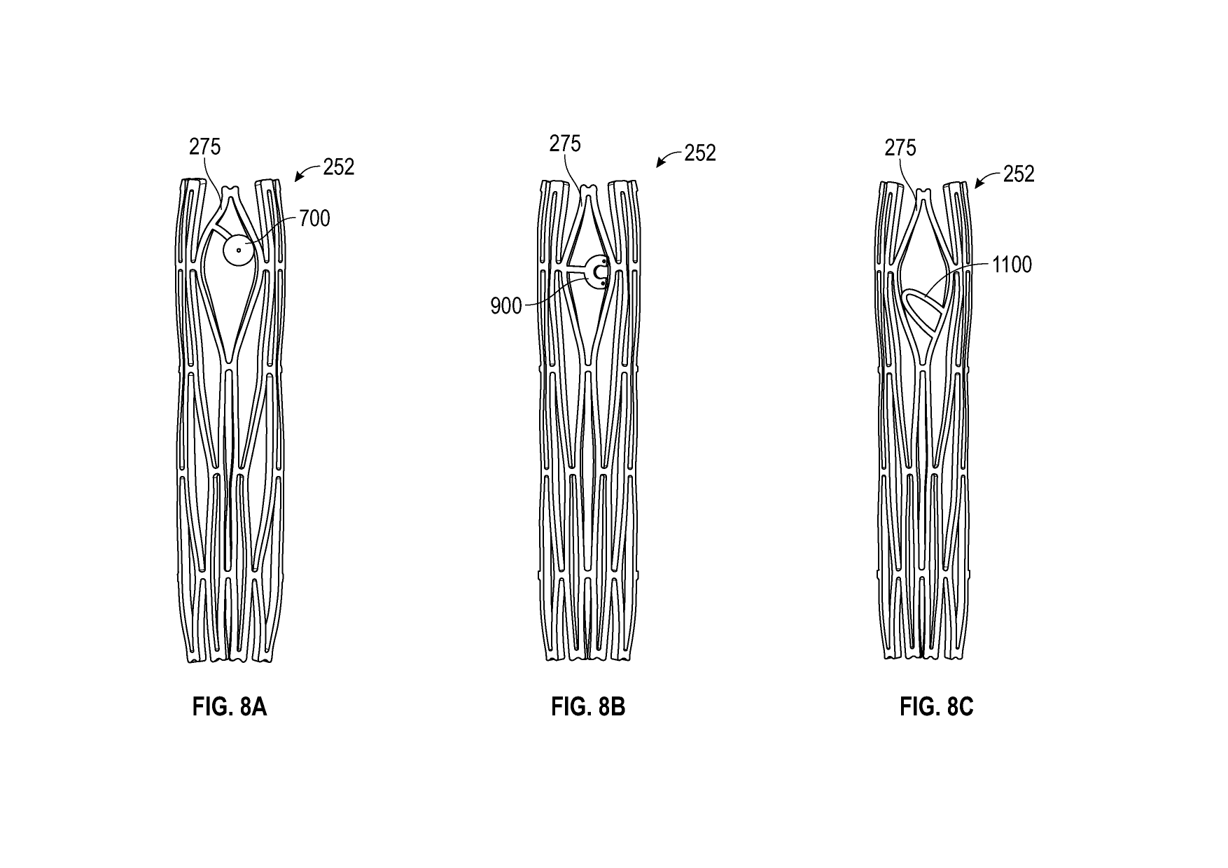

In some examples, an integral marker can extend from an interior edge of a strut in an inflow portion of a cell (that is, a portion of the cell oriented toward the inflow end of the prosthetic valve). For example, FIG. 7A shows the cell 275 including an integral marker 1000. The marker 1000 can extend from the interior edge 202 of the strut 272d, which is in an inflow portion of the cell 275, and can be disconnected from other struts of the cell (272a, 272b, 272c). As illustrated in FIG. 7A, in the present example, no portion of the marker extends from an exterior edge 210 of the cell 275. The marker 1000 can include a main body comprising a partial ovular member 1004 (for example, a half ovular member). The marker 1000 can further include linear extensions 1006, wherein the extensions 1006 extend from the interior edge 202 to a flat region of the partial ovular member 1004. The member 1004 can extend into one side of the open area the cell 275 near a junction portion between the struts 272b, 272d, thereby giving the marker 1000 an asymmetrical orientation within the cell 275. Thus, the marker 1000 can be a directional indicator (indicating a left or a right-handed direction of the frame 252).

In another example, FIG. 7B shows the cell 275 including an integral marker 1100. Specifically, the marker 1100 can extend from the interior edge 202 of the struts 272, which is in an inflow portion of the cell 275, and can be disconnected from other struts of the cell (272a, 272b, 272c). As illustrated in FIG. 7B, in the present example, no portion of the marker 1100 extends from an exterior edge 210 of the cell 275. The marker 1100 can include a main body comprising an elongated D-shaped member 1104. The D-shaped member 1104 can intersect with the interior edge 202 at two locations of the strut 272d. The D-shaped member 1104 can extend into the open area the cell 275 at an angle toward a center of the cell, thereby giving the marker 1100 an asymmetrical orientation within the cell 275. Thus, the marker 1100 can be a directional indicator (indicating a left or a right-handed direction of the frame 252).

The integral markers disclosed herein can be configured to facilitate or enable axial elongation of the cell 275 during radial compression of the frame 252. For example, the markers can have a size, a shape, a location, and/or an orientation within the commissure-supporting cells that enables the cell to be compressed into an axially elongated shape when the frame (and the prosthetic valve) is in a radially compressed state. FIGS. 8A-8C show the frame 252 in a radially compressed state and the cell 275 in an axially elongated state. As can be seen therein, each of the exemplary integral markers 700, 900, 1100 can have a size, a shape, a location, and/or an orientation within the cell 275 that allows the cell to axially elongate when the frame 252 is in the radially compressed state. For example, each of the integral markers 700, 900, 1100 can be configured such that (that is, can have a size, a shape, a location, and/or an orientation within the cell 275) a width of the integral marker being less than or equal to a width of the cell 275, when the cell is in the axially elongated state, at the location of the integral marker.

Turning to FIGS. 9A–11, in some examples, the frame 252 can include three cells 275 (which can be commissure-supporting cells) circumferentially spaced around the frame in the outflow row of cells. In some examples, the three cells 275 are disposed at a 120⁰ angle relative to each other. In some examples, each of the three cells 275 can include more than one radiopaque marker. For examples, each of the cells 275 can include a first marker 1200 and a second marker 1300.

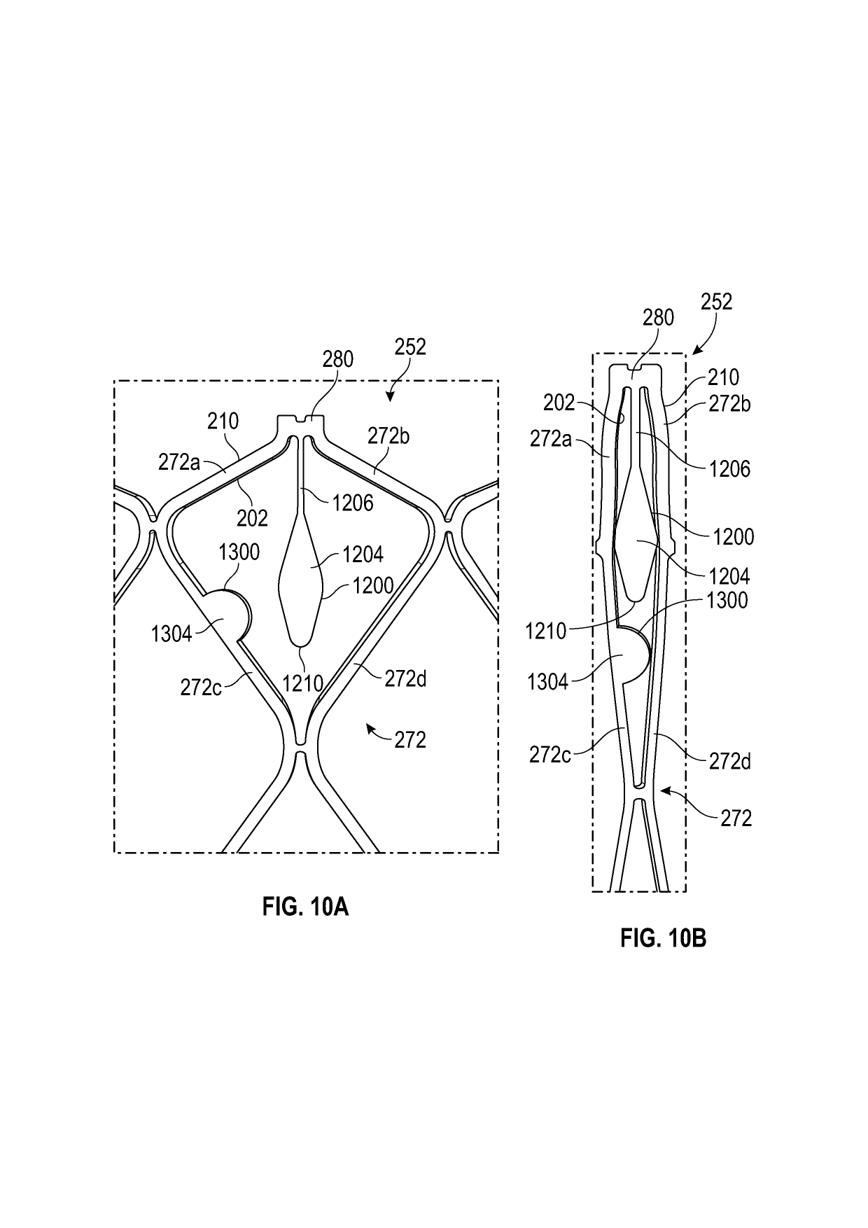

As best illustrated in FIG. 10A, the first marker 1200 can extend from the interior edge 202 of the cell 275, at a junction between struts 272a and 272b, and can be disconnected from other struts of the cell (272c, 272d). In some examples, the junction between struts 272a and 272b can be an apex 280 of the frame 252. In the illustrated example, no portion of the marker 1200 extends from an exterior edge 210 of the cell 275. The first marker 1200 can include a main body comprising a diamond-shaped member 1204 having a rounded distal end 1210. The marker 1200 can further include a linear neck portion 1206 that is narrower than the main body, wherein the linear neck portion 1206 extends between the interior edge 202 and the diamond-shaped member 1204. The integral marker 1200 can extend parallel to a central axis of the frame and/or can bisect at least a portion of the cell 275. Thus, in some examples, the integral marker 1200 has a paddle-shape that is symmetrical (non-directional). In some examples, the first marker 1200 can be asymmetrical. For example, the main body can a tab portion extending from a side of the main body or the main body can be asymmetrical in shape and/or can extend from the neck portion towards one side of the cell, thereby making the marker asymmetrical (directional). In some examples, the marker 1200 can additionally include an aperture at a free end of the linear member 204 (that is, at an opposing end of the member 204 relative to where it extends from the interior edge 202).

The second marker 1300 can extend from the interior edge 202 of the cell 275 at the strut 272c and can be disconnected from other struts of the cell (272a, 272b, 272d). As illustrated in FIGS. 10A and 10B, no portion of the marker 1300 extends from the exterior edge 210 of the cell 275. The marker 1300 can include a main body comprising a partial or semi-circular member 1304 (for example, a 1/2 portion of a circle) having a continuously curved outer edge extending from the strut 272c. In some examples, the marker 1300 can additionally include an aperture. In the present example, the semi-circular member 1304 has an overall symmetrical shape, however, as it extends into one side of the open area the cell 275, the marker 1300 has an asymmetrical orientation within the cell 275. Thus, the marker 1300 can be a directional indicator (indicating a left or a right-handed direction of the frame 252).

In some examples, the frame 252 including the integral markers 1200 and 1300 in each of the commissure-supporting cells 275 can be utilized for identifying commissure-supporting cells 275 of the frame 252 and for identifying a forward-most commissure-supporting cell 275 and/or a rear-most commissure-supporting cell 275 during an implantation procedure. For example, the symmetrical integral markers 1200 can be utilized for identifying a center portion of commissure-supporting cells 275 and can be aligned to commissures of a native valve and/or a previously implanted prosthetic valve during an implantation procedure. Further, in some examples, the asymmetrical integral markers 1300 can be utilized for identifying which of the commissure-supporting cells is forward and/or rearward of the other commissure-supporting cell, such as, for example, when viewed under fluoroscopy. For example, as best illustrated in FIG. 9A, in the forward-most cell 275, the marker 1300 is disposed on the lower left portion of the cell, whereas in the cells 275 rearward of the forward-most cell 275, the marker 1300 is disposed in the lower right portion of the cell. Identification of the forward and rearward positions of the commissure-supporting cells 275 can further enable alignment of the commissure-supporting cells with the commissures of a native valve and/or a previously implanted prosthetic valve during an implantation procedure. For example, the forward-most commissure-supporting cell 275 can be aligned to the forward-most commissure of the native valve and/or the previously implanted prosthetic valve during an implantation procedure.

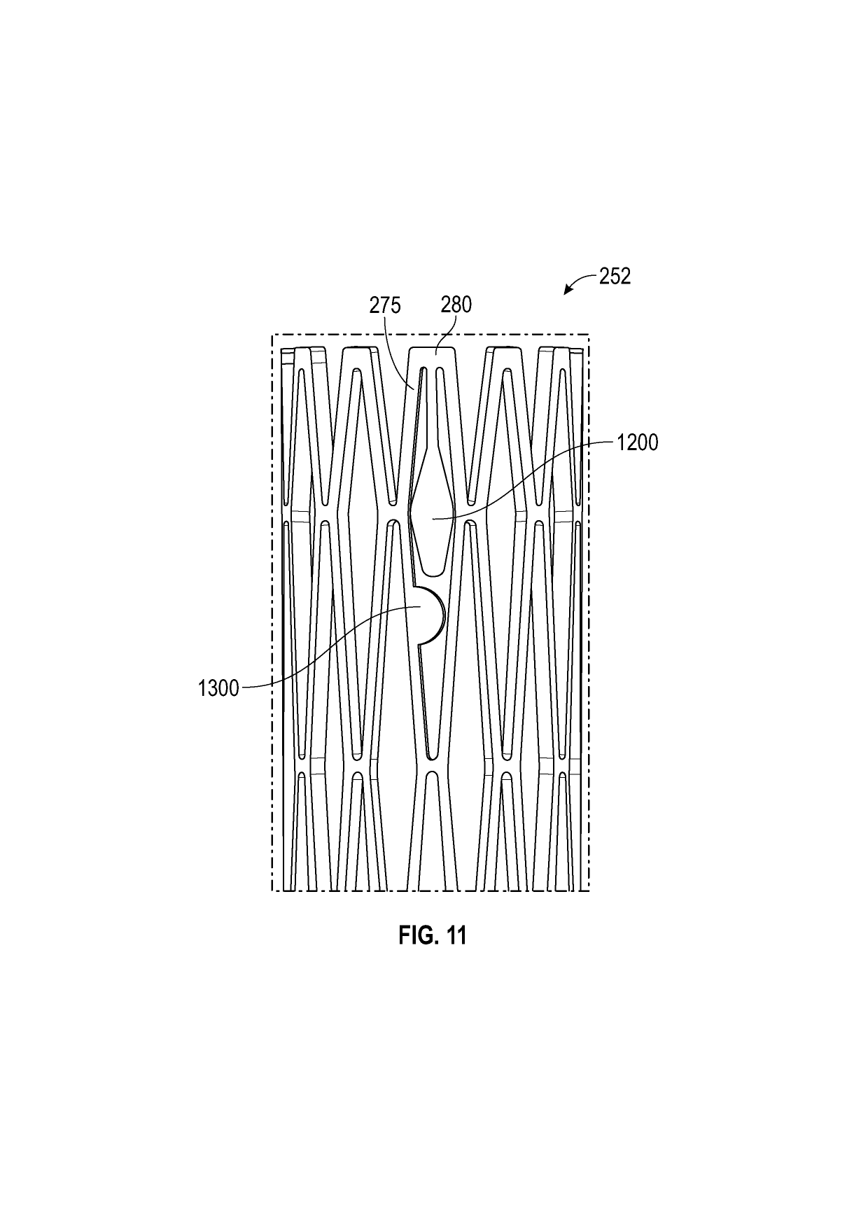

As discussed above, the integral markers can be configured to facilitate or enable axial elongation of the cell 275 during radial compression of the frame 252. For example, the markers 1200, 1300 can have a size, a shape, a location, and/or an orientation within the commissure-supporting cells that enables the cell 275 to be compressed into an axially elongated shape when the frame 252 is in a radially compressed state. Further, the markers 1200, 1300 can have a size, a shape, a location, and/or an orientation that prevents the markers from contacting each other when the cell 275 is compressed into an axially elongated shape. FIGS. 10B and 11 show the frame 252 in a radially compressed state and the cell 275 in an axially elongated state. As can be seen therein, each of the exemplary integral markers 1200 and 1300 can have a size, a shape, a location, and/or an orientation within the cell 275 that allows the cell to axially elongate when the frame 252 is in the radially compressed state and prevents contact between the integral markers 1200 and 1300. In some examples, each of the integral markers 1200 and 1300 can be configured such that (that is, can have a size, a shape, a location, and/or an orientation within the cell 275) a width of the integral marker is less than or equal to a width of the cell 275, when the cell is in the axially elongated shape/state, at the location of the integral marker. In some examples, each of the integral markers 1200 and 1300 can be configured such that (that is, can have a size, a shape, a location, and/or an orientation within the cell 275) the marker 1200 is disposed above the marker 1300 with a gap disposed therebetween when the cell is in the axially elongated shape/state.

In some examples, the integral markers disclosed herein can include one or more features of another integral marker. In some examples, the integral markers disclosed herein can have a different orientation within a cell and/or can extend from a different portion of the interior edge of the cell. As noted above, in some examples, the integral marker is positioned within a commissure-supporting cell. In some examples, the integral marker can be located in another cell to indicate a location of the commissure-supporting cell, such as within one or more cells that are directly upstream of a commissure-supporting cell, or within one or more cells that are circumferentially adjacent to and/or located at a side of a commissure-supporting cell. In some examples, a frame can include one or more additional outflow row of cells downstream of the commissure-supporting cells, which can include one or more integral markers (in such examples, the commissure-supporting cells do not form the outflow end of the frame).

In some examples, a frame can include an integral marker at one or more of the commissure-supporting cells. In some examples, a frame can include an integral marker at each of its commissure-supporting cells (for example, as shown in the example of FIGS. 9A and 9B). Thus, in a prosthetic valve having three commissures and three commissure-supporting cells (such as, for example, the prosthetic valve 50), the frame can have three integral markers (each located within a respective commissure-supporting cell). In some examples, each of the integral marker can be identical. In some examples, one or more of the integral markers can be different (for example, having a different shape and/or orientation) relative to other integral markers of the frame.

In some examples, a frame can include any combination of markers disclosed herein in the same cell or in different cells of the frame. For example, a frame can include a marker 1200 and any one of markers 200, 300, 400, 500, 600, 700, 800, 900, 1000, 1100 in the same cell(s) or in different cells.

As discussed above, in some examples, the frame illustrated in FIGS. 3A-11 can have one or more of the frame 52 features shown in FIGS. 1A-2 and/or can be utilized in a prosthetic valve having one or more of the valve 50, 150 features discussed above with respect to FIGS. 1A-2. In some examples, the frame of FIGS. 3A-11 can have different features from the frame 52, and/or can be utilized in a prosthetic valve with different features from the prosthetic valves 50, 150.

In some examples, a prosthetic valve can comprise a marker (for example, any of markers 200, 300, 400, 500, 600, 700, 800, 900, 1000, 1100, 1200, 1300) separately formed from the frame 252 and subsequently connected to the frame, such as by welding.

### Delivery Techniques

For implanting a prosthetic valve having one or more integral markers within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve (for example, via alignment of the integral markers with commissures of the native valve) and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve having one or more integral markers within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve having one or more integral markers within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve having one or more integral markers (mounted on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient’s vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve comprising: an annular frame comprising a plurality of interconnected struts that form a plurality of cells; and a valve structure disposed within an interior space of the frame and having an inflow end and an outflow end, the valve structure comprising a plurality of leaflets, each of the leaflets joined to an adjacent leaflet at the outflow end of the valve structure to form a commissure therebetween, the commissure attached to the frame at a commissure-supporting cell of the plurality of cells; wherein the commissure-supporting cell defines an interior opening; and wherein the frame comprises an integral marker, the integral marker extending from an interior edge of the commissure-supporting cell into the interior opening.

Example 2. The prosthetic heart valve of any example disclosed herein, particularly example 1, wherein the integral marker does not extend outside of the interior opening.

Example 3. The prosthetic heart valve of any example disclosed herein, particularly examples 1 or 2, wherein the integral marker does not extend from an exterior edge of the commissure-supporting cell.

Example 4. The prosthetic heart valve of any example disclosed herein, particularly examples 1–3, wherein the commissure-supporting cell comprises an outflow apex portion, and wherein the integral marker extends from the interior edge at the outflow apex portion.

Example 5. The prosthetic heart valve of any example disclosed herein, particularly examples 1–3, wherein the integral marker extends from the interior edge at a junction portion of two interconnected struts at one side of the commissure-supporting cell.

Example 6. The prosthetic heart valve of any example disclosed herein, particularly examples 1–5, wherein the integral marker has an asymmetrical shape.

Example 7. The prosthetic heart valve of any example disclosed herein, particularly example 6, wherein the integral marker comprises a main body comprising at least one of a linear member or a circular member, the main body having a projection extending outwardly therefrom towards a side portion of the commissure-supporting cell.

Example 8. The prosthetic heart valve of any example disclosed herein, particularly examples 1–6, wherein the integral marker has an asymmetrical orientation within the commissure-supporting cell.

Example 9. The prosthetic heart valve of any example disclosed herein, particularly examples 1–6 or 8, wherein the integral marker includes a neck portion and a main body portion, and wherein the neck portion extends between the interior edge and the main body portion and has a narrower width than the main body portion.

Example 10. The prosthetic heart valve of any example disclosed herein, particularly example 9, wherein the main body portion comprises a C-shaped member.

Example 11. The prosthetic heart valve of any example disclosed herein, particularly example 9, wherein the main body portion comprises a circular member.

Example 12. The prosthetic heart valve of any example disclosed herein, particularly example 9, wherein the main body portion comprises a half-circular member.

Example 13. The prosthetic heart valve of any example disclosed herein, particularly example 9, wherein the main body portion comprises a half-ovular member.

Example 14. The prosthetic heart valve of any example disclosed herein, particularly examples 9–13, wherein the neck portion extends at an angle towards a center portion of the interior opening of the commissure-supporting cell.

Example 15. The prosthetic heart valve of any example disclosed herein, particularly examples 1–6 or 8, wherein the integral marker comprises a curved member.

Example 16. The prosthetic heart valve of any example disclosed herein, particularly example 15, wherein the curved member extends from a first strut in an outflow portion of the commissure-supporting cell to a second strut at in inflow portion of the commissure-supporting cell.

Example 17. The prosthetic heart valve of any example disclosed herein, particularly examples 1–16, wherein the integral marker is sized and shaped to enable axial elongation of the commissure-supporting cell when the frame is a radially compressed state.

Example 18. The prosthetic heart valve of any example disclosed herein, particularly examples 1–17, wherein the integral marker includes one or more apertures.

Example 19. The prosthetic heart valve of any example disclosed herein, particularly examples 1–18, wherein the integral marker is configured to enable identification of the commissure-supporting cell during one or more of an implantation procedure or a post-implantation procedure.

Example 20. A prosthetic heart valve comprising: an annular frame comprising a plurality of interconnected struts forming a plurality of cells, wherein each of the cells comprises an interior opening; and a valve structure disposed within an interior space of the frame and having an inflow end and an outflow end, the valve structure comprising a plurality of leaflets; wherein selected ones of the cells include an integral marker extending from an interior edge of the respective selected cell and into the interior opening thereof; wherein no portion of the integral marker extends outside of the interior opening; and wherein the integral marker is configured to enable identification of the respective selected cell during one or more of an implantation procedure or a post-implantation procedure.

Example 21. The prosthetic heart valve of any example disclosed herein, particularly example 20, wherein the selected ones of the cells are in an outflow row of cells of the frame.

Example 22. The prosthetic heart valve of any example disclosed herein, particularly examples 20 or 21, wherein the selected ones of the cells are commissure-supporting cells, each of the commissure-supporting cells supporting a commissure formed between adjacent ones of the leaflets of the valve structure.

Example 23. The prosthetic heart valve of any example disclosed herein, particularly examples 20–22, wherein the integral marker is a directional indicator.

Example 24. The prosthetic heart valve of any example disclosed herein, particularly example 23, wherein the integral marker has an asymmetrical shape.

Example 25. The prosthetic heart valve of any example disclosed herein, particularly examples 23 or 24, the integral marker has an asymmetrical orientation within the respective selected cell.

Example 26. The prosthetic heart valve of any example disclosed herein, particularly examples 20–25, wherein the integral marker includes a neck portion and a main body portion, and wherein the neck portion extends between the interior edge and the main body portion and has a narrower width than the main body portion.

Example 27. The prosthetic heart valve of any example disclosed herein, particularly example 26, wherein the neck portion extends from the interior edge at one of a first strut in an outflow portion of the respective selected cell or a second strut in an inflow portion of the respective selected cell, and wherein the neck portion extends at an angle toward a center portion of the interior opening of the respective selected cell.

Example 28. The prosthetic heart valve of any example disclosed herein, particularly example 26, wherein the neck portion extends from the interior edge at a junction portion of two interconnected struts at one side of the respective selected cell and extends towards a center portion of the interior opening of the respective selected cell.

Example 29. The prosthetic heart valve of any example disclosed herein, particularly examples 20–25, wherein the integral marker comprises a curved member, the curved member having to points of intersection with the interior edge of the respective selected cell.

Example 30. The prosthetic heart valve of any example disclosed herein, particularly examples 20–25, wherein the integral marker comprises a crescent-shaped member continuous with a junction portion of two interconnected struts at one side of the respective selected cell.

Example 31. A prosthetic heart valve comprising: an annular frame comprising a plurality of interconnected struts forming a plurality of cells, wherein each of the cells comprises an interior opening; and a valve structure disposed within an interior space of the frame and having an inflow end and an outflow end, the valve structure comprising a plurality of leaflets; wherein selected ones of the cells each include one or more integral markers extending from an interior edge of the respective selected cell and into the interior opening thereof; wherein no portion of the integral marker extends outside of the interior opening; and wherein the one or more integral markers are configured to enable identification of the respective selected cells during one or more of an implantation procedure or a post-implantation procedure.

Example 32. The prosthetic heart valve of any example disclosed herein, particularly example 31, wherein the selected ones of the cells are in an outflow row of cells of the frame.

Example 33. The prosthetic heart valve of any example disclosed herein, particularly examples 31 or 32, wherein the selected ones of the cells are commissure-supporting cells, each of the commissure-supporting cells supporting a commissure formed between adjacent ones of the leaflets of the valve structure.

Example 34. The prosthetic heart valve of any example disclosed herein, particularly examples 31–33, wherein a first integral marker of the one or more the integral markers is a directional indicator that has one or more of an asymmetrical shape or an asymmetrical orientation within a respective one of the selected cells.

Example 35. The prosthetic heart valve of claim 34, wherein a second integral marker of the one or more the integral markers is a symmetrical marker including a neck portion and a main body portion, and wherein the neck portion extends between the interior edge and the main body portion and has a narrower width than the main body portion.

Example 36. The prosthetic heart valve of claim 35, wherein the neck portion extends from the interior edge at a junction portion of two interconnected struts at an apex of the respective selected cell and extends towards a center portion of the interior opening of the respective selected cell.

Example 37. The prosthetic heart valve of claim 35 or claim 36, wherein the first and second integral markers are configured such that one or more of a size, a shape, a location, or an orientation of each of the first and second integral markers enables axial elongation of the commissure-supporting cell when the frame is a radially compressed state.

Example 38. The prosthetic heart valve of claims 35–37, wherein the first integral markers in the selected cells are configured to enable identification of a forward-most one of the selected cells relative to others of the selected cells during one or more of an implantation procedure or a post-implantation procedure.

Example 39. A prosthetic heart valve comprising: an annular frame comprising a plurality of interconnected struts forming a plurality of cells, wherein each of the cells comprises an interior opening; and a valve structure disposed within an interior space of the frame and having an inflow end and an outflow end, wherein the valve structure comprises a plurality of leaflets and each of the leaflets is joined to an adjacent leaflet at the outflow end of the valve structure to form a commissure therebetween, and wherein the commissures are attached to the frame at commissure-supporting cells of the plurality of cells; wherein each of the commissure-supporting cells includes a symmetrical integral marker that bisects as least a portion of the respective commissure-supporting cell and a directional marker, each of the symmetrical integral marker and the directional marker extending from an interior edge of the respective commissure-supporting cell and into the interior opening thereof, wherein the directional marker has at least one of an asymmetrical shape or an asymmetrical orientation within the respective commissure-supporting cell; and wherein the one or more integral markers are configured to enable, for each of the commissure-supporting cells, identification of whether each of the respective commissure-supporting cell is in one or a forward orientation or a rearward orientation during one or more of an implantation procedure or a post-implantation procedure.

Example 40. A method comprising sterilizing the prosthetic heart valve, apparatus, and/or assembly of any one of example 1–39.

Example 41. A prosthetic heart valve of any one of examples 1–39, wherein the prosthetic heart valve is sterilized.

The features described herein with regard to any example can be combined with other features described in any one or more of the other examples, unless otherwise stated. For example, any one or more of the features of one frame can be combined with any one or more features of another frame. As another example, any one or more features of one integral marker can be combined with any one or more features of another integral marker.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A prosthetic heart valve comprising:
an annular frame comprising a plurality of interconnected struts that form a plurality of cells; and
a valve structure disposed within an interior space of the frame and having an inflow end and an outflow end, the valve structure comprising a plurality of leaflets, each of the leaflets joined to an adjacent leaflet at the outflow end of the valve structure to form a commissure therebetween, the commissure attached to the frame at a commissure-supporting cell of the plurality of cells;
wherein the commissure-supporting cell defines an interior opening; and
wherein the frame comprises an integral marker, the integral marker extending from an interior edge of the commissure-supporting cell into the interior opening.

2. The prosthetic heart valve of claim 1, wherein the integral marker does not extend outside of the interior opening.

3. The prosthetic heart valve of claim 1, wherein the integral marker does not extend from an exterior edge of the commissure-supporting cell.

4. The prosthetic heart valve of claim 1, wherein the commissure-supporting cell comprises an outflow apex portion, and wherein the integral marker extends from the interior edge at the outflow apex portion.

5. The prosthetic heart valve of claim 1, wherein the integral marker extends from the interior edge at one side of the commissure-supporting cell.

6. The prosthetic heart valve of claim 1, wherein the integral marker has an asymmetrical shape.

7. The prosthetic heart valve of claim 1, wherein the integral marker has an asymmetrical orientation within the commissure-supporting cell.

8. The prosthetic heart valve of claim 1, wherein the integral marker includes a neck portion and a main body portion, and wherein the neck portion extends between the interior edge and the main body portion and has a narrower width than the main body portion.

9. The prosthetic heart valve of claim 1, wherein the integral marker comprises a curved member, wherein the curved member extends from a first strut in an outflow portion of the commissure-supporting cell to a second strut at an inflow portion of the commissure-supporting cell.

10. The prosthetic heart valve of claim 1, wherein the integral marker is configured such that one or more of a size, a shape, a location, or an orientation of the integral marker enables axial elongation of the commissure-supporting cell when the frame is a radially compressed state.

11. The prosthetic heart valve of claim 1, wherein the integral marker is configured to enable identification of the commissure-supporting cell during one or more of an implantation procedure or a post-implantation procedure.

12. A prosthetic heart valve comprising:
an annular frame comprising a plurality of interconnected struts forming a plurality of cells, wherein each of the cells comprises an interior opening; and
a valve structure disposed within an interior space of the frame and having an inflow end and an outflow end, the valve structure comprising a plurality of leaflets;
wherein at least one selected cell of the plurality of cells includes one or more integral markers extending from an interior edge of the respective selected cell and into the interior opening thereof;
wherein no portion of the integral marker extends outside of the interior opening; and
wherein the one or more integral markers are configured to enable identification of the selected cell during one or more of an implantation procedure or a post-implantation procedure.

13. The prosthetic heart valve of claim 12, wherein the selected cell is in an outflow row of cells of the frame.

14. The prosthetic heart valve of claim 12, wherein the selected cell is a commissure-supporting cell supporting a commissure of the leaflets of the valve structure.

15. The prosthetic heart valve of claim 12, wherein a first integral marker of the one or more the integral markers is a directional indicator that has one or more of an asymmetrical shape or an asymmetrical orientation within the selected cell.

16. The prosthetic heart valve of claim 15, wherein a second integral marker of the one or more the integral markers is a symmetrical marker including a neck portion and a main body portion, and wherein the neck portion extends between the interior edge and the main body portion and has a narrower width than the main body portion.

17. The prosthetic heart valve of claim 16, wherein the neck portion extends from the interior edge at a junction portion of two interconnected struts at an apex of the selected cell and extends towards a center portion of the interior opening of the selected cell.

18. The prosthetic heart valve of claim 16, wherein the first and second integral markers are configured such that one or more of a size, a shape, a location, or an orientation of each of the first and second integral markers enables axial elongation of the at least one selected cell when the frame is a radially compressed state.

19. The prosthetic heart valve of claim 16, wherein the at least one selected cell comprises a plurality of selected cells, each including respective first and second integral markers, wherein the first integral markers in the selected cells are configured to enable identification of a forward-most one of the selected cells relative to others of the selected cells during one or more of an implantation procedure or a post-implantation procedure.

20. A prosthetic heart valve comprising:
an annular frame comprising a plurality of interconnected struts forming a plurality of cells, wherein each of the cells comprises an interior opening; and
a valve structure disposed within an interior space of the frame and having an inflow end and an outflow end, wherein the valve structure comprises a plurality of leaflets and each of the leaflets is joined to an adjacent leaflet at the outflow end of the valve structure to form a commissure therebetween, and wherein the commissures are attached to the frame at commissure-supporting cells of the plurality of cells;
wherein each of the commissure-supporting cells includes a symmetrical integral marker that bisects as least a portion of the respective commissure-supporting cell and a directional marker, each of the symmetrical integral marker and the directional marker extending from an interior edge of the respective commissure-supporting cell and into the interior opening thereof, wherein the directional marker has at least one of an asymmetrical shape or an asymmetrical orientation within the respective commissure-supporting cell; and
wherein the symmetrical integral marker and the directional marker are configured to enable, for each of the commissure-supporting cells, identification of whether each of the respective commissure-supporting cell is in one or a forward orientation or a rearward orientation during one or more of an implantation procedure or a post-implantation procedure.
